(19) United States
(12) Patent Application Publication
(10) Pub. No.: US 2005/0101996 A1
(43) Pub. Date: May 12, 2005

(54) Apparatus and methods of medical intervention in an electromagnetic gauge symmetry space higher than the U(1) symmetry

(75) Inventors: William A. Tiller, Payson, AZ (US); Walter E. Dibble JR., Payson, AZ (US)

Correspondence Address: MORRIS MANNING & MARTIN LLP, 1600 ATLANTA FINANCIAL CENTER, 3343 PEACHTREE ROAD, NE, ATLANTA, GA, 30326-1044, US

(73) Assignee: Ditron LLC, Excelsior, MN (US)

(21) Appl. No.: 10/985,329

(22) Filed: Nov. 10, 2004

Related U.S. Application Data
(63) Continuation-in-part of application No. 10/453,710, filed on Jun. 3, 2003.

(51) Int. Cl. A61N 1/00

(52) U.S. Cl. 607/2

(57) ABSTRACT

A method for therapeutic treatment of a living subject in a limited physical space, wherein the limited physical space is characterized by a series of symmetry states having a ground symmetry state and at least one higher symmetry state. In one embodiment, the method includes the steps of providing an intention imprinted electronic device adapted for a specific condition of the living subject, activating the intention imprinted electronic device in the limited physical space for a period of time so as to transform the limited physical space from the ground symmetry state to a higher symmetry state, and placing the living subject in the limited physical space for a period of time for therapeutic treatment of the specific condition of the living subject.

### CROSS-REFERENCE TO RELATED PATENT APPLICATION

This application is a continuation-in-part of U.S. patent application Ser. No. 10/453,710, filed Jun. 3, 2003, entitled “APPARATUS AND METHODS OF MEASURING A THERMODYNAMIC POTENTIAL OF A LIMITED PHYSICAL SPACE CHARACTERIZED BY A SERIES OF SYMMETRY STATES,” by William A. Tiller and Walter E. Dibble, Jr., which status is pending and is incorporated herein by reference in its entirety.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference. In terms of notation, hereinafter, “[n]” represents the nth reference cited in the list of references. For example, [9] represents the 9th reference cited in the list of references, namely, W. A. Tiller, W. E. Dibble, Jr. and M. J. Kohane, Conscious Acts of Creation: The Emergence of a New Physics, (Pavior Publishing, Walnut Creek, Calif., 2001).

### FIELD OF THE INVENTION

The present invention generally relates to apparatus and methods for therapeutic treatment of at least one living subject, and in particular to utilization of an intention imprinted electronic device to transform a limited physical space from the ground symmetry state to a higher symmetry state for therapeutic treatment of the at least one living subject.

### BACKGROUND OF THE INVENTION

Most people including scientists may never give the concept of electromagnetic (hereinafter “EM”) gauge symmetry much thought, although they may know that the macroscopic properties of materials are almost completely determined by their electric and magnetic behavior. If the topic ever comes up in conversation between colleagues, it is generally considered to be in the domain of interest of particle physicists or relativity theorists. However, perhaps they might remember that it may enter the concept of the “Big Bang” theory, a cosmological theory that the universe began in a state of compression to infinite density and has been expanding since some particular instant that marked the origin of the universe, in an important way. They might recall that a short time after the initiation event, and before to after quarks form in the incredibly hot “fireball”, the universe evolves to successively lower thermodynamic free energy states via step by step changes in gauge symmetry, eventually passing through a SU(2) gauge symmetry state on its way to a U(1) gauge symmetry state, which, as discussed in more detail infra, is often referred to as the U(1) EM gauge symmetry state. Thereafter, the free energy of the U(1) gauge symmetry state continues to be lowered by a set of phase transitions, as known to people skilled in the art, from plasma phase to gas phase then to liquid phase and then to solid phase as cooling continues. From the foregoing, one might deduce that, if one could raise the EM gauge symmetry state associated with an environment such as a laboratory, the properties of materials in that environment would change. In fact, they should be EM gauge symmetry state specific. The challenge is how to change the EM gauge symmetry state of a limited physical space and identify a measurable physical property associated with the change.

A variety of studies [1-6] have indicated that human intention can give rise to robust changes in physical phenomena. Particularly, under proper protocols, an aspect of human consciousness, i.e., a specific human intention, can be encoded into a simple electronic device, which, when activated, can raise a limited physical space such as a laboratory to a higher EM gauge symmetry state so as to robustly alter measured properties of both inanimate and animate materials [5, 9] in complete accordance with the encoded human intention, because properties of materials are related to EM gauge symmetry of the surrounding environment.

For example, having activated an electronic device that is encoded with a specific intention of raising a pH of purified water by one full pH unit in a laboratory, one has observed the pH changes of the purified water in the range of 0.5 to 1.0 pH units (a factor of approximately 10 in H+concentration) in the laboratory [12, 13]. When the electronic device that is encoded with a specific intention of reducing the pH of the purified water by one full pH unit has been activated in the laboratory, a reduction of the pH of the purified water by one full pH unit has been observed in the laboratory [13]. Both experiments are conducted with no chemical additions and measurement uncertainty in the range of ±0.01 pH unit. For a specific intention of increasing the in vivo adenosine tri-phosphate/adenosine diphosphate (hereinafter “ATP/ADP”) ratio in the cells of developing fruit fly larvae so that they become significantly more fit and thus exhibit reduced larval development time to the adult fly stage, a significant change (about 25%) in both the ATP/ADP ratio and the larval development time changed has been recorded with excellent statistics having p<0.001 [6, 9], where p, with a value ranging from zero to one, corresponds to a probability value of a statistical hypothesis test. For a specific intention of increasing the in vitro thermodynamic activity of the liver enzyme alkaline phosphatase (hereinafter “ALP”), a significant amount with an increase of about 25% has been achieved with statistics excellent having p<0.001[6, 9].

These experimental data, along with other data [1-6, 9, 12-14], show that, far from being simply things that communicate ideas and feelings, human thoughts are energy units, which can be expressed in waves and particles, and interact with and/or condition their surrounding environment through focused intentions, which indicates that there is a connection between energy and human consciousness [1-6]. Having considered that there is a connection between mass and energy, quantified by Einstein, applicants conclude that the following interact/reaction equation exists in a physical reality:
MASS [symbol] ENERGY [symbol] CONSCIOUSNESS. (1)
The equation suggests that, like mass and energy, human consciousness and energy are reciprocally convertible, and thus the human consciousness is ultimately measurable and quantifiable.

Human health is generally thought to depend on the integrity of the body structure. The integrity of the body structure depended on a relatively stable state of equilibrium (i.e., homeostasis) between the different but interdependent structural parts. In turn, homeostasis depended on a host of organic and inorganic chemicals. Whenever the body does not function properly, structural defects arising out of chemical imbalances are blamed. To cure this problem and restore the needed homeostasis of the body, some sophisticated chemical complexes, i.e., drugs, may be used to trigger the body's defense and repair mechanisms. This approach leads to what can be called “chemical medicine” with its many strengths and weaknesses.

Human consciousness has been successfully utilized to condition a physical space to raise an EM gauge symmetry of the physical space to an EM gauge symmetry higher than the U(1) gauge symmetry so as to alter EM field properties of the physical space such that when an object exposes to the conditioned physical space, the interaction of the field of the conditioned physical space with the object will modify properties of the object in complete accordance with a specific human intention. The object can be either a biological object or a non-biological object. This may suggest that human intention is capable of changing chemical and physical properties of the body of a living subject (a human or an animal), via raising a local EM gauge symmetry state of their surrounding space. Thus, if reproducible procedures for raising an EM gauge symmetry of a physical space to an EM gauge symmetry higher than the U(1) gauge symmetry in accordance with a human intention of curing a particular malady of a living subject could be developed, great clinical relevance would be gained.

Therefore, a heretofore unaddressed need exists in the art to address the aforementioned deficiencies and inadequacies.

### SUMMARY OF THE INVENTION

In one aspect, the present invention relates to a method for therapeutic treatment of a living subject in a limited physical space. The limited physical space is characterized by a series of symmetry states having a ground symmetry state and at least one higher symmetry state, where the ground symmetry state is characterized by a U(1) electromagnetic gauge symmetry, and the at least one higher symmetry state is characterized by a symmetry that is not a U(1) electromagnetic gauge symmetry. In one embodiment, the at least one higher symmetry state is characterized by a SU(2) symmetry. The limited physical space includes a space substantially confined in three dimensions. In one embodiment, the confined space has an interior space confined by a stationary structure. In another embodiment, the confined space has an interior space confined by a structure that is stationary or in motion. Examples of structures having confined space include offices, houses, vehicles, operating rooms, patient rooms, and doctors offices in association with hospitals, laboratories, etc. Some of the structures are stationary like offices and rooms associated with a building. Some of the structures can be both stationary and in motion. For example, a vehicle is a stationary structure when it does not move, and is a structure in motion when it moves.

In one embodiment, the method includes the steps of providing an intention imprinted electronic device adapted for a specific condition of the living subject, activating the intention imprinted electronic device in the limited physical space for a period of time so as to transform the limited physical space from the ground symmetry state to a higher symmetry state, and placing the living subject in the limited physical space for a period of time for therapeutic treatment of the specific condition of the living subject.

The method, in one embodiment, further includes the step of keeping the intention imprinted electronic device tuned to the specific condition of the living subject for at least a portion of the period of time during which the living subject is placed in the limited space. Moreover, the method includes the step of monitoring response of the living subject in the limited physical space at the higher symmetry state for evaluation of the therapeutic treatment for the living subject. Additionally, the method has the steps of maintaining the limited physical space at the higher symmetry state, and monitoring the magnitude of a thermodynamic free energy related to the limited physical space to provide feedback to the maintaining step. The thermodynamic free energy of the limited physical space comprises a proton magnetoelectrochemical potential energy. Furthermore, the method includes the step of conditioning the limited space with an information encoded radiation generated from the intention imprinted electronic device, where the information encoded radiation includes one of an electric field signal, a magnetic field signal, and a combination thereof.

In another aspect, the present invention relates to an apparatus for therapeutic treatment of a living subject in a limited physical space, wherein the limited physical space is characterized by a series of symmetry states having a ground symmetry state and at least one higher symmetry state. In one embodiment, the apparatus has an intention imprinted electronic device adapted for a specific condition of the living subject. When the intention imprinted electronic device is activated in the limited physical space for a period of time, the limited physical space is transformed from the ground symmetry state to a higher symmetry state. The apparatus further has means for monitoring the magnitude of a thermodynamic free energy related to the limited physical space as a function of time to determine the activation time period of the intention imprinted electronic device for therapeutic treatment of a living subject in the limited physical space. The monitoring means, in one embodiment, has a meter for measuring the magnitude of a thermodynamic free energy related to the limited physical space.

In one embodiment, the intention imprinted electronic device includes a single-oscillator circuit adapted for generating signals corresponding to a specific condition of the living subject. In another embodiment, the intention imprinted electronic device includes a circuit having a plurality of oscillators adapted for generating signals corresponding to a specific condition of the living subject.

In yet another aspect, the present invention relates to a method for creating an intention imprinted electronic device for therapeutic treatment of a living subject in a limited physical space, wherein the limited physical space is characterized by a series of symmetry states having a ground symmetry state and at least one higher symmetry state. In one embodiment, the method includes the step of providing an electronic device having an electronic circuit with at least one oscillator. The electronic circuit further has an input node for receiving an input voltage, a switch coupled to the input node for changing the electronic circuit between a first state and a second state, and an indication means to indicate the state of the electronic circuit. In one embodiment, the switch is movable between an on position and an off position. When the switch is at the on position, the electronic circuit is in the first state, and when the switch is at the off position, the electronic circuit is in the second state. The at least one oscillator and the indication means are electrically coupled to the switch, respectively. The indication means, in one embodiment, has a light emitting diode.

The method further includes the step of imprinting a predetermined intention into the electronic device when the electronic device is at the first state so as to create an intention imprinted electronic device, wherein the predetermined intention is designed for therapeutic treatment of a living subject in a limited physical space. In one embodiment, the imprinting step includes the steps of intentionally cleansing the electronic device, broadcasting in the limited physical space a statement corresponding to the predetermined intention designed for therapeutic treatment of the living subject in the limited physical space on the cleansed electronic device, interfacing the image of the cleansed electronic device and the stated predetermined intention for a first period of time to encode information corresponding to the stated predetermined intention into the electronic device to allow the at least one oscillator to generate imprint signals containing information corresponding to the stated predetermined intention, intentionally sealing the electronic device for a second period of time, and repeating the broadcasting step, the interfacing step and the sealing step till the strength of imprint signals reaches a predetermined threshold.

In one embodiment, the imprinting step is performed by a group of people who are capable of entering a predetermined meditative state of consciousness and focusing the predetermined meditative state for a period of time. In one embodiment, the first time period is longer than the second time period, where the first time period is in a range of about 10 to 60 minutes, and preferably in the range of about 10 to 20 minutes, and the second time period is in a range of about 1 to 15 minutes, and preferably in the range of about 2 to 10 minutes.

In a further aspect, the present invention relates to a method for creating an intention imprinted electronic device for therapeutic treatment of at least one living subject by a group of people who are capable of entering a predetermined meditative state of consciousness. In one embodiment, the method includes the step of providing an electronic device having an electronic circuit with at least one oscillator. The electronic circuit has an input node for receiving an input voltage, a switch coupled to the input node for changing the electronic circuit between a first state and a second state, and an indication means to indicate the state of the electronic circuit. The at least one oscillator and the indication means are electrically coupled to the switch, respectively. The method also includes the step of focusing a first predetermined meditative state of consciousness on an electronic device for a first period of time to cleanse the electronic device. The first predetermined meditative state of consciousness comprises a statement of an intention of cleansing the electronic device. The method further includes the step of focusing a second predetermined meditative state of consciousness on the cleansed electronic device for a second period of time to encode information of the second predetermined meditative state of consciousness into the cleansed electronic device. The second predetermined meditative state of consciousness comprises a statement of an intention of therapeutic treatment for the at least one living subject. Moreover, the method further includes the step of focusing a third predetermined meditative state of consciousness on the encoded electronic device for a third period of time to seal the encoded electronic device. The third predetermined meditative state of consciousness comprises a statement of an intention of sealing the encoded information into the encoded electronic device and minimizing leakage of the encoded information from the encoded electronic device. In one embodiment, the electronic device includes an electronic circuit having at least one oscillator.

In yet a further aspect, the present invention relates to a method for therapeutic treatment of a group of living subjects remotely from a limited physical space, wherein each of the group of living subjects is identified by a name and an address of the corresponding living subject, and the limited physical space is characterized by a series of symmetry states having a ground symmetry state and at least one higher symmetry state. In one embodiment, the method includes the step of providing a first intention imprinted electronic device and a second intention imprinted electronic device in the limited physical space, wherein the first intention imprinted electronic device is adapted for a specific condition of the group of living subjects, and the second intention imprinted electronic device is adapted for broadcasting specific information of the limited physical space on each of the group of living subjects remotely. Furthermore, the method includes the steps of activating the first intention imprinted electronic device in the limited physical space for a period of time so as to transform the limited physical space from the ground symmetry state to a higher symmetry state related to the specific condition of the group of living subjects, and activating the second intention imprinted electronic device in the limited physical space for a period of time so as to tune the limited physical space to broadcast the higher symmetry state on one of the group of living subjects whose name and address are presented in the limited space. Moreover, the method includes the step of presenting a name and an address of each of the group of living subjects in the limited space iteratively for therapeutic treatment of the group of living subjects.

The method, in one embodiment, further includes the step of monitoring response of each of the group of the living subjects remotely for evaluation of the therapeutic treatment for the corresponding living subject. Additionally, the method includes the steps of maintaining the limited physical space at the higher symmetry state, and monitoring the magnitude of a thermodynamic free energy related to the limited physical space to provide feedback to the maintaining step.

In another aspect, the present invention relates to an apparatus for therapeutic treatment of a group of living subjects remotely from a limited physical space, wherein each of the group of living subjects is identified by a name and an address of the corresponding living subject, and the limited physical space is characterized by a series of symmetry states having a ground symmetry state and at least one higher symmetry state. In one embodiment, the apparatus has a display adapted for presenting a name and an address of each of the group of living subjects iteratively in the limited space. The apparatus further has a first intention imprinted electronic device adapted for creating a specific condition of the limited physical space. When the first intention imprinted electronic device is activated in the limited physical space for a period of time, the limited physical space is transformed from the ground symmetry state to a higher symmetry state related to the specific condition of the group of lining subjects. Moreover, the apparatus has a second intention imprinted electronic device adapted for broadcasting the higher symmetry state on each of the group of living subjects. When the second intention imprinted electronic device is activated in the limited physical space for a period of time, the limited physical space is tuned to broadcast the higher symmetry state on one of the group of the living subjects whose name and address are presented on the display in the limited space. Additionally, the apparatus has means for monitoring the magnitude of a thermodynamic free energy related to the limited physical space as a function of time, to determine the activation time period of the first intention imprinted electronic device. In one embodiment, the monitoring means has a meter for measuring the magnitude of a thermodynamic free energy related to the limited physical space.

In one embodiment, the first intention imprinted electronic device has a circuit with at least one oscillator. The second intention imprinted electronic device also has a circuit with at least one oscillator.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

FIG. 1 shows schematically a fiber-bundle space.

FIG. 2 shows a fiber-bundle space of FIG. 1 with a self-coherent system.

FIG. 3 shows an electronic circuit diagram of an intention imprinted electronic device: (a) a single-oscillator embodiment, and (b) a three-oscillator embodiment.

FIG. 4 shows state anxiety indices of a control group of living subjects and a therapeutic treated group of living subjects according to one embodiment of the present invention.

FIG. 5 schematically shows a perspective view of a portable electronic device according to one embodiment of the present invention.

### DETAILED DESCRIPTION OF THE INVENTION

The present invention is more particularly described in the following examples that are intended as illustrative only since numerous modifications and variations therein will be apparent to those skilled in the art. Various embodiments of the invention are now described in detail. Referring to the drawings, like numbers indicate like parts throughout the views. As used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which has no influence on the scope of the invention. Additionally, some terms used in this specification are more specifically defined below.

### Definitions

The terms used in this specification generally have their ordinary meanings in the art, within the context of the invention, and in the specific context where each term is used.

Certain terms that are used to describe the invention are discussed below, or elsewhere in the specification, to provide additional guidance to the practitioner in describing the apparatus and methods of the invention and how to make and use them. For convenience, certain terms may be highlighted, for example using underlining, italics and/or quotation marks. The use of highlighting has no influence on the scope and meaning of a term; the scope and meaning of a term is the same, in the same context, whether or not it is highlighted. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms may be provided. A recital of one or more synonyms does not exclude the use of other synonyms. The use of examples anywhere in this specification, including examples of any terms discussed herein, is illustrative only, and in no way limits the scope and meaning of the invention or of any exemplified term. Likewise, the invention is not limited to various embodiments given in this specification.

As used herein, “about” or “approximately” shall generally mean within 20 percent, preferably within 10 percent, and more preferably within 5 percent of a given value or range. Numerical quantities given herein are approximate, meaning that the term “about” or “approximately” can be inferred if not expressly stated.

The term “consciousness,” as defined in the Webster Dictionary [7], refers to “the state of being consciousness; awareness of one's own feelings and what is happening around one, etc.” and/or “the totality of one's thoughts; feelings and impressions.” Psychology, on the other hand, talks about the conscious and the unconscious states of mind. While Norretranders [8] indicates that humans are mostly unconscious since the amount of information entering human bodies and proceeding to the brain is about 50 million bits per second but the information channel capacity of human conscious brain is experimentally found to be less than about 50 bits per second. However, the term “consciousness” in equation (1) is meant to differentiate a unique quality of nature that is ultimately measurable and quantifiable. As used herein, the term “consciousness” refers to the meaning of consciousness associated with equation (1).

The term “intention,” as defined in the Webster Dictionary [7], refers to (i) “the act or fact of intending; determination to do a specified thing or act in a specified manner,” (ii) “an instrument, such as a concept, to knowing and referring to a thing as it exists in the mind,” and/or (iii) “the general word implying a having something in mind as a plan or design, or referring to a plan held in mind.” As used herein, the term “intention” refers to the meaning of definition (iii). That is, the intention is an aspect of consciousness.

As used herein, the term “electromagnetic gauge symmetry” shall generally mean a characteristic state of a physical system related to the conservation of electric charge. People are generally familiar with symmetries that are geometrical in appearance, like the hexagonal (6-fold) symmetry of a snowflake or 4-fold symmetry of a cube. The charge symmetry of electric materials is non-geometric in that, for an ensemble of electric dipoles when the individual charges are suddenly reversed in sign, the energy of the ensemble as a whole is unchanged (“invariant”) so the forces also remain unchanged. The same type of behavior occurs for magnetic dipoles and electromagnetic fields in general. The character of the symmetry that makes Maxwell's theory of electromagnetism a gauge theory is that the electric field is invariant with respect to the addition or subtraction of an arbitrary overall electric potential. A related symmetry can be demonstrated for the phase of an electron wave in the quantum theory of electrons since any phase angle can be added to or subtracted from the electron field and the results of all experiments will remain invariant. This is the essential ingredient found in the EM gauge symmetry or U(1) gauge symmetry.

As used herein, the term “U(1) EM gauge symmetry level” refers to an EM gauge symmetry state in conventional physical reality where materials include electric monopole, electric dipole and magnetic dipole that construct substances, standard Maxwell's equations of EM apply, all particles and waves have subluminal velocities, the governing algebra is Abelian (i.e., XY−YX=0, where X and Y are fields) and the vacuum space between the fundamental particles comprising atoms and molecules is isotropic. Magnetic monopoles are not accessible from the U(1) EM gauge symmetry level of reality. In addition, the EM potentials and fields are mostly vectors given in terms of their amplitudes and phase angles. For the U(1) EM gauge symmetry state, the phase angle can take on all possible values in a plane so that these phase values can be interpreted as an angular coordinate in a 2-dimensional space with two orthogonal coordinates. The internal space of the U(1) EM gauge symmetry state thus looks like the interior of a flat ring and the coordinate of each point in the internal space is just the phase value itself. The internal space is very appropriately called a “fiber” by mathematicians.

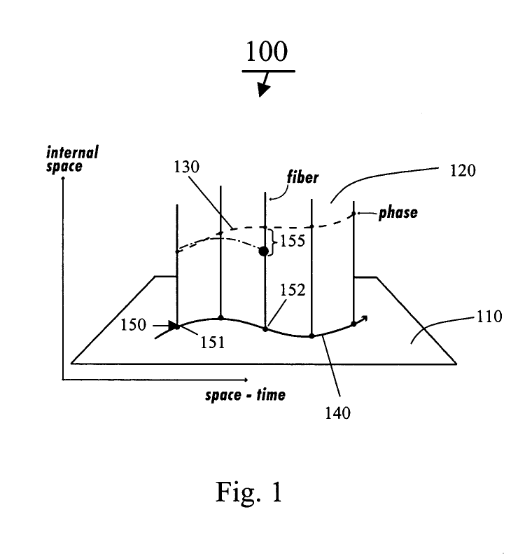

FIG. 1 shows a space 100 that combines an internal space with space-time [10]. In the space 100, the spatial location of a particle 150 is given by a coordinate point in the horizontal, space-time plane 110 and the orientation of the particle 150 in the internal space 120 is specified by angular coordinates in the space 100. As the particle 150 moves through the space-time space 110, it traces out a path 130 in the internal space 120 above a space-time trajectory 140. When there is no external gauge potential acting on the particle, the internal space path 130 is completely arbitrary. When the particle interacts with an external gauge field, the path 130 in the internal space 120 is a continuous curve determined by the gauge potential. In mathematical jargon, the space formed by the union of 4-dimensional space-time with an internal space is called a “fiber bundle” space.

The term “higher EM gauge symmetry levels,” as used herein, refers to EM gauge symmetry states different from the U(1) gauge symmetry state. All higher EM gauge symmetry states exhibit higher thermodynamic free energy per unit volume than the U(1) EM gauge symmetry state does because, during cooling of the universe from the supposed “big bang” state, it went from higher EM gauge symmetry states to lower EM gauge symmetry states so the thermodynamic free energy per unit volume also decreased. SU(2) is an EM gauge symmetry state higher than U(1) EM gauge symmetry state.

It has been discovered that the nuclear force is independent from charges. That is the strength of the nuclear interaction between a proton and a neutron, or between two protons or two neutrons is the same. Charge independence is formulated as a new symmetry principle through an introduction of concept of isotopic spin, and defined as an invariance of the nuclear force under a new SU(2) isotopic-spin rotation group in the analogy to the law for conservation of angular momentum. For example, the proton and the neutron can be considered as the “up” and “down” states of an abstract isotopic spin, respectively, similar to the ordinary spin states of an electron. In the analogy to the electromagnetic potential, Yang and Mills postulated a new potential field of the isotopic spin. However, the great complexity of the SU(2) isotopic-spin rotation group, compared to the U(1) phase group, means that the Yang-Mills potential is quite different from that of the EM field.

Another example that requires the use of the SU(2) gauge symmetry state is the situation where both electric and magnetic monopoles, and thus both electric and magnetic currents, naturally coexist. This leads to non-Abelian fields which obey the commutation relationship XY−YX≠0. Consequently, the governing electrodynamic equations are non-Maxwell's equations with non-Abelian algebra [11], which are called the Maxwell-Barrett equations. In order to move from the higher degrees of freedom state of the SU(2) EM gauge symmetry state to the lower degrees of freedom of the U(1) EM gauge symmetry state, a symmetry breaking transformation is required.

The internal space defined for the SU(2) gauge symmetry state is more complicated than that for the U(1) gauge symmetry state because it describes rotation in a three-dimensional (herein after “3D”) space. For the SU(2) gauge symmetry state, values of three angles which specify the rotation can be considered as the coordinates of a point inside an abstract 3D space. Thus, the internal symmetry space of the SU(2) gauge symmetry state looks like the interior of a 3D sphere so that, for the SU(2) gauge symmetry state, one would have to attach a sphere at each space-time point in FIG. 1.

As used herein, the term “symmetry breaking” shall generally mean that the situation of which this symmetry is broken is characterized by a lower symmetry than the situation of which this symmetry is not broken. The breaking of a certain symmetry does not imply that no symmetry is present. In group theory terms, this means that the initial symmetry group is broken to one of its subgroups.

A well-known example of symmetry breaking in atomic physics is the Zeeman effect in the hydrogen atom, where the forces are the U(1) EM force and the symmetry involved is the 3D rotational symmetry of the Coulomb potential. This symmetry is broken by turning on an external magnetic field that is aligned along one direction of space [10]. Before turning on the magnetic field, the wave function for the electron is spherically symmetric, while the spherical symmetry is broken after the magnetic field is turned on. After symmetry breaking, the dependence of the wave function on azimuthal angle is no longer arbitrary and the degeneracy of the energy levels of the hydrogen atom is lifted. In other words, by introducing the external magnetic field, the symmetry is broken from a complete rotational symmetry in the 3D space to a smaller set of rotations about the direction of the external magnetic field.

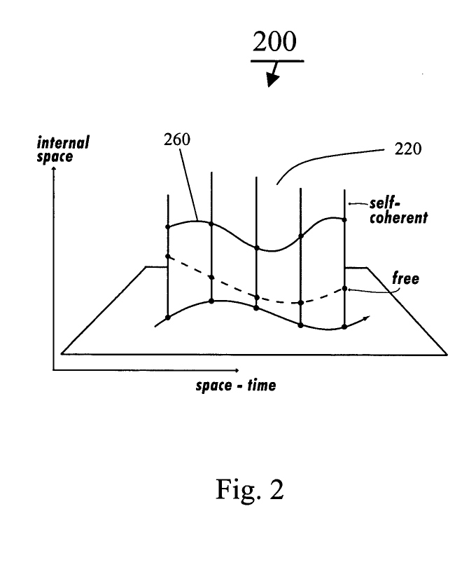

As shown in FIG. 1, for a point-like particle 150, any change 155 in the internal direction between different space-time points 151 and 152 is completely determined by the minimal coupling to the gauge potential field, the internal direction is free to rotate in any way required by the gauge field so no constraints exist on such an internal direction. In order to break the symmetry imposed by its interaction with the gauge field, the particle must somehow determine its own direction in the internal symmetry space independent from the requirement of the external gauge field. At the same time, the particle must still be minimally coupled to the gauge field to preserve the invariance of its Lagrangian [10]. This means that the wave function of the particle must have an intrinsic relative phase factor that is different than that illustrated in FIG. 1. In turn, this means that a new type of physical system that interacts with the gauge field, called self-coherent system, is introduced, as shown in FIG. 2, where the phase of the self-coherent system traces out a path 260 in the internal space 220 that is one of a free point-like particle in the same gauge field [10]. The gauge field itself must undergo certain changes in order to adjust its phase requirements to that of the self-coherent system. This is the actual meaning of gauge symmetry breaking. The gauge field is “broken” from the free particle form to the self-coherent form.

Deltrons deltron coupling and magnetoelectric energy: Abundant and costly experiments have been carried out in search of magnetic monopoles by many laboratories around the world and no magnetic monopole has been observed in such laboratories having the U(1) EM gauge symmetry level. From these observations, one may conclude that either (a) magnetic monopoles do not exist in nature or (b) magnetic monopoles exist but are not accessible from the U(1) EM gauge symmetry level. However, a series of experiments recently conducted by the applicants in a conditioned space has shown that (i) magnetic monopoles, or at least monopole images, are accessible from such a conditioned laboratory space [9], and (ii) very low frequency oscillations, in a range of frequency from 10⁻³ Hz to 10⁻² Hz, appear on digital readouts from certain types of electronic measurement equipment.

If one postulates that magnetic monopoles, functioning in the physical vacuum, are inaccessible via measurement apparatus that are constrained to the U(1) EM gauge symmetry level because such magnetic monopoles are constrained to only travel at speeds greater than or equal to the velocity of the light, c, deltrons allow magnetic monopoles to become accessible because the deltrons are a higher dimensional particle beyond the constraints of space-time relativity theory so the deltrons can travel at a speed slower than c, to interact with electric particles, and faster than c, to interact with magnetic particles, and thus couple one to the other. It is this deltron coupling that allows conventional electrical instrumentation to extend its functional capacities beyond the light barrier at a speed equal to c and into a superluminal range via deltron-deltron interactions.

Such deltron-deltron interactions allow magnetoelectric (hereinafter “ME”) phenomena to become instrumentally accessible. In this case, magnetic currents generate electric fields and these ME waves all travel at a speed greater than c, in contrast to electric currents generating magnetic fields so that electromagnetic waves all travel at a speed lees than c. For EM waves, conventional photons are involved while, for ME waves, a new type of photons is required to be present. One of the important characteristics of ME waves is that ME waves can be modulated by information content of specific human intentions, which, via deltron coupling, allow coupling of these modulated ME waves to EM waves so that all this becomes instrumentally accessible to conventional U(1) gauge instrumentation.

Thermodynamic free energy per unit volume and operational thermodynamic variables: At the U(1) EM gauge symmetry level of physical reality, the thermodynamic free energy per unit volume of a material is most simply defined in terms of three extensive variables P, T and Cⱼ, which are pressure, temperature and concentration of j-species (j=0, 1, 2, . . . m), respectively. When the U(1) EM gauge symmetry level of a system is raised to a higher EM gauge symmetry level, the system is in a state which involves greatly enhanced deltron activation, the thermodynamic free energy per unit volume of the system is raised and more extensive variables are now involved in describing this expanded thermodynamics. These additional variables are understood to have at least (a) intention, (b) consciousness, and (c) degree of deltron activation.

As used herein, the term “conditioned space” refers to a space exhibiting a higher EM gauge symmetry state than the U(1) EM gauge symmetry state, and a higher thermodynamic free energy per unit volume than that of the same space at the U(1) EM gauge symmetry state.

As used herein, “Xᵢ”, i=1, 2, 3, . . . , represents a person that is identifiable with a name and a residence address and involved in certain examples of practicing the present invention.

The human Qi/prana pump: Experimental kinesiological techniques indicate that the human acupuncture meridian/chakra system is at a sufficiently higher EM gauge symmetry state than the U(1) EM gauge symmetry state that magnetic monopoles in the physical vacuum are accessible to the human acupuncture meridian/chakra system. This allows human intention to modulate the ME radiation streams in the bio-field of the human which, in turn, stimulates, enlivens and energizes the U(1) EM gauge symmetry level of the physical body, which may have significant health benefits to the human body. It also allows such health benefits to be transferred to the human body from a conditioned space wherein the human body spends a sufficient period of exposure time.

### Overview of the Invention

The present invention, in one aspect, relates to a method for therapeutic treatment of a living subject in a limited physical space. The limited physical space is characterized by a series of symmetry states having a ground symmetry state and at least one higher symmetry state. The living subject can be either a human or an animal.

In one embodiment, the method includes the following steps: at first, an intention imprinted electronic device (hereinafter “IIED”) adapted for a specific condition of the living subject is provided. In one embodiment, the specific condition of the living subject corresponds to an illness of the living subject. Secondly, the IIED is activated in the limited physical space for a period of time so as to transform the limited physical space from the ground symmetry state to a higher symmetry state. The ground symmetry state is characterized by a U(1) electromagnetic gauge symmetry, and the at least one higher symmetry state is characterized by a symmetry that is not a U(1) electromagnetic gauge symmetry. In one embodiment, the at least one higher symmetry state is characterized by a SU(2) symmetry. The IIED activation, in one embodiment, is to turn power on the IIED. The period of time of the IIED activation, in one embodiment, is about 2 to 3 months. And then, the living subject is placed in the limited physical space for a period of time for therapeutic treatment of the specific condition of the living subject. The period of time for therapeutic treatment is determined by the thermodynamic free energy of the limited physical space, in terms of the magnitude of a proton magnetoelectrochemical potential energy, Ψ_{H}_{⁺}, in the limited physical space. In one embodiment, water is utilized to continuously monitor the proton magnetoelectrochemical potential energy Ψ_{H}_{⁺}, by measuring water pH values as a function of time in the limited physical space. If the magnitude of the proton magnetoelectrochemical potential energy is below a predetermined threshold value, the IIED needs to be re-imprinted. In one embodiment, the re-imprinting time interval is in a range of about 3 to 12 months.

In alternative embodiments, other liquid and/or solid materials, such as (a) water containing various kinds of gels of a wide range of gel concentrations from about 0% to 100%, with or without added electrolytes and surfactants, (b) water containing colloid particles of a wide range of sizes, chemical types and concentrations, with or without added electrolytes and surfactants, (c) any types of liquid electrolytes, and (d) any types of solid electrolytes, can also be used to monitor the proton magnetoelectrochemical potential energy in the limited physical space.

Since thermodynamics of all chemical species present in water, other liquid or solid materials is electrochemically measurable, the magnetoelectrochemical potential energy Ψⱼ can be obtained for any species, j, and utilized to gain a measure of an EM gauge symmetry higher than the U(1) symmetry. In practice, the utility of a measured Ψⱼ for this purpose depends upon the kinetics of equilibration for the j-species reaction in an electrochemical system. For example, for j=H⁺, the equilibration kinetics are fairly rapid. For j=e⁻, for the electron as measured in a typical oxidation/reduction reaction, the equilibration kinetics may be appreciately slow but still useful for measuring of Ψⱼ For j=O₂, as in the oxygen solubility reaction, or for j=Cu⁺⁺⁺, as in the cupric ion solubility reaction, the equilibration kinetics may be slow but still very useful for measuring of Ψⱼ.

The limited physical space includes a space substantially confined in three dimensions. The confined space has an interior space that is at least partially confined by a stationary structure. In one embodiment, the stationary structure includes any and all man-made structures from temperatory to permanent buildings, private home to apartment complex, business establishment to manufacturing factories, sports facilities to art displays, clinics to hospitals, bedrooms, laboratories, and the like. The stationary structure, in another embodiment, may include nature structures such as cave, cavern, and the like. The confined space also comprises the interior space confined by a structure that is capable of being stationary or in motion, which includes any and all man-made vehicles such as automobiles, bus, train, ship, air plane, submarine, space shuttle, and the like. Thus, when a man-made vehicle is not in motion such as a parked automobile, the man-made vehicle is a stationary structure. When the same man-made vehicle is in motion such as a moving automobile, the man-made vehicle is a structure in motion. The confined space can have exposure to the space outside the structure, but that exposure can be controlled. For example, a room of a building or a building itself can communicate with the space outside the room or the building through doors and windows, which can be opened or closed.

The present invention also discloses a method for therapeutic treatment of a group of living subjects remotely from a limited physical space. Each of the group of living subjects is identifiable and identified by a name and an address of the corresponding living subject. In one embodiment, the method includes the step of providing a first IIED and a second IIED in the limited physical space. The first IIED is adapted for a specific condition of the group of living subjects. The specific condition of the group of living subjects may correspond to an illness. The second IIED is adapted for broadcasting specific information of the limited physical space generated from the first IIED on each of the group of living subjects remotely. Furthermore, the method includes the steps of activating the first IIED in the limited physical space for a period of time so as to transform the limited physical space from the ground symmetry state to a higher symmetry state related to the specific condition of the group of living subjects, and activating the second IIED in the limited physical space for a period of time so as to tune the limited physical space to broadcast the higher symmetry state of the limited physical space on one of the group of living subjects whose name and address are presented in the limited space. In one embodiment, the period of time of activation of the first IIED is about 2 to 3 months. In one embodiment, a water pH measuring apparatus is utilized to continuously monitor the water proton magnetoelectrochemical potential energy Ψ_{H}_{⁺}, as a function of time in the limited physical space so as to monitor the magnitude of a proton magnetoelectrochemical potential energy related to the higher symmetry state in the limited physical space.

Moreover, the method includes the step of presenting a name and an address of each of the group of living subjects iteratively in the limited space for therapeutic treatment of the specific condition of the group of living subjects. In one embodiment, a list of names and addresses of the group of living subjects is scrolled continuously through a computer in the limited physical space.

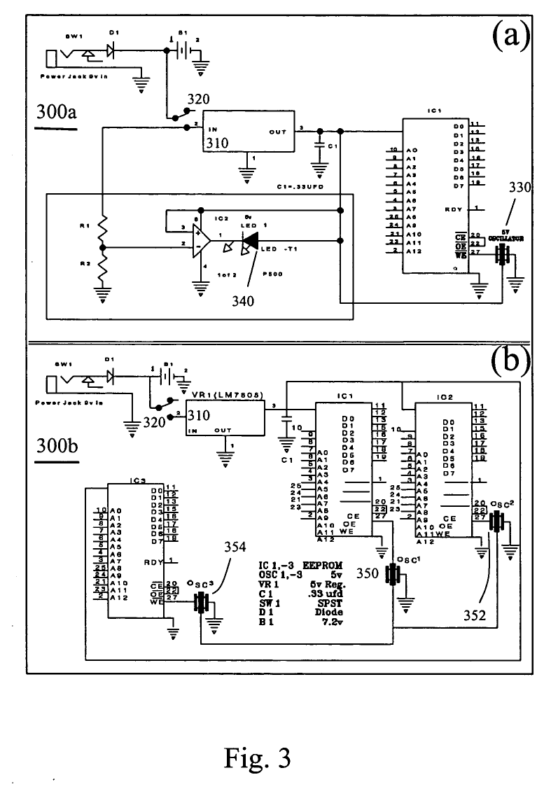

Although almost any type of objects and/or materials can be intention imprinted, an electronic circuit constitutes a sufficiently efficient device for practicing this invention. In one embodiment, the electronic circuit includes a single-oscillator. The electronic circuit, in another embodiment, includes a three-oscillator. FIGS. 3a and 3b show exemplary circuit diagrams for the single-oscillator and the multi-oscillator embodiments for practicing the present invention, respectively. In one embodiment as shown in FIG. 3a, an electronic circuit 300a for practicing the present invention has an input node 310 for receiving an input voltage, a switch 320 coupled to the input node 310 for changing the electronic circuit 300a between a first state and a second state, and an indication means 340 to indicate the state of the electronic circuit 300a. The switch 320 is movable between an on position and an off position. When the switch 320 is at the on position, the electronic circuit 300a is in the first state, which is the on state where a current flows through the circuit 300a and the oscillator 330 generates and outputs EM signals to the surrounding environment and/or receives and stores EM signals from the surrounding environment, and when the switch 320 is at the off position, the electronic circuit 300a is in the second state, which is the off state, where there is no current in the circuit 300a. The indication means 340, in one embodiment, has a light emitting diode (hereinafter “LED”). FIG. 3b shows another electronic circuit 300b for practicing the present invention that has multi-oscillator embodiment. More specifically, the circuit 300b has a first oscillator 350, a second oscillator 352, and a third oscillator 354 electrically coupled in parallel. Moreover, the electronic circuit 300a or 300b can be enclosed in a casing that is substantially formed with materials that are transparent to EM signals such as plastics.

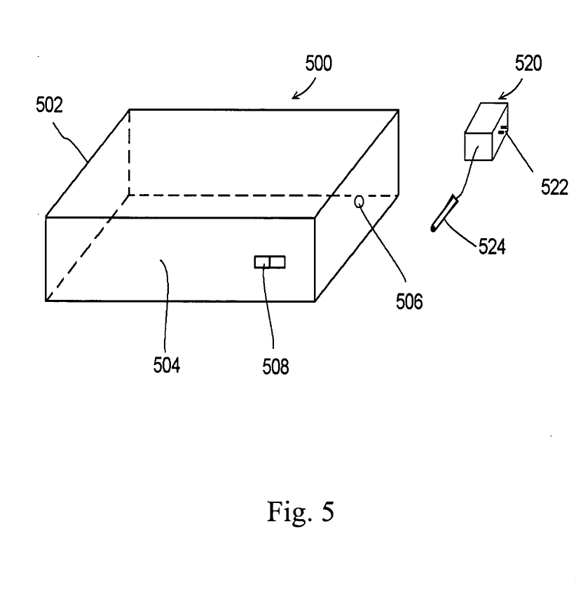

The circuit 300a or 300b can be powered by a DC source such as battery or batteries or an AC source. As shown in FIG. 5, a portable electronic device 500 has a body portion 502 defining a volume 504 for receiving an electronic circuit such as an electronic circuit 300a or an electronic circuit 300b therein. The body portion 502 has an insertion hole 506 adapted for receiving a conducting plug 524 of a power converter 520 therein. The power converter 520 has contact means 522 that can be received in an outlet (not shown) such that an AC current can be introduced into the power converter 520 through the contact means 522 and converted into a DC current by the power converter 520, which can then be provided to an electric circuit contained in the portable electric device 500 through the engagement of the conducting plug 524 with the insertion hole 506 formed on the body portion 502. A switch 508 can be used to turn the portable electric device 500 on or off. In one embodiment, a portable electronic device having a single oscillator is sized about 4″×2½″×¾″ or the like, and a portable electronic device having three oscillators is sized about 7″×2½″×¾″ or the like.

A reproducible procedure for imprinting a predetermined intention into the electronic device is now detailed. The imprinting process is performed by a group of people who are capable of entering a predetermined meditative state of consciousness and focusing the predetermined meditative state for a period of time. To start the imprinting process, an electronic device as set forth above is placed on a table around which the group of people sits, with power on, the group of people enters a meditative state of cleansing the surrounding environment to create a “sacred” or clean space in preparation for the imprinting process. Then one of the group of people gives a directive to enter a first predetermined meditative state of consciousness, all of the group of people enters the first predetermined meditative state of consciousness and focuses the first predetermined meditative state of consciousness on the electronic device for a first period of time to mentally cleanse the electronic device. In one embodiment, the directive to enter the first predetermined meditative state of consciousness includes a statement of an intention of cleansing the electronic device. The first period of time is in general about 3 to 4 minutes. It is understood that the state of consciousness associated with a human being may take the form of EM signals, which can interface with the electronic device as set forth above.

After the electronic device is cleansed, one of the group of people reads a statement of a specific intention as a directive to enter a second predetermined meditative state of consciousness for the group of people, then, all of the group of people enters the second predetermined meditative state of consciousness, focuses the second predetermined meditative state of consciousness on the cleansed electronic device for a second period of time to encode information of the second predetermined meditative state of consciousness into the cleansed electronic device, and then abruptly releases the second predetermined meditative state of consciousness. Here the term “abruptly releases” indicates a state that each of the group of people takes the corresponding action substantially at the same time without any intent to hold it back. The second period of time, in one embodiment, is about 10 to 15 minutes. In one embodiment, the specific intention includes an intention of medical intervention for a living subject, and/or a group of living subjects. Now the electronic device is considered as an “encoded electronic device.”

Next, a directive is given to enter a third predetermined meditative state of consciousness of sealing the encoded electronic device, then, all of the group of people enters a third predetermined meditative state of consciousness and focuses the third predetermined meditative state of consciousness on the encoded electronic device for a third period of time to seal the encoded electronic device, and then abruptly releases the third predetermined meditative state of consciousness. The third predetermined meditative state of consciousness includes a statement of an intention of sealing the encoded information into the encoded electronic device and minimizing leakage of the encoded information from the encoded electronic device. The third period of time, in one embodiment, is about 5 minutes.

This reproducible procedure is repeated until the imprint signal is sufficiently strong.

An example of a statement of a specific intention to be imprinted into an electronic device is: “To activate the indwelling consciousness of the laboratory space in order to significantly raise the EM gauge symmetry state of that space. The special characteristic or ‘tuning’ of this IIED-created state is to increase the pH of the experimental water located in that space by one full pH unit above that of the control condition, determined by conventional theory for the U(1) EM gauge symmetry state of that general environment. This means that the H⁺ ion content of this higher EM gauge symmetry state water is decreased by a factor of 10 relative to that of the U(1) EM gauge symmetry state.” Effects of the statement of the specific intention on a pH of purified water have been observed at several sites and different times, and the pH increases of the purified water in the range of 0.5 to 1.0 pH units have been achieved with a measurement accuracy of about ±0.01 pH units for such purified water in equilibrium with air, with no chemical additions [9].

The present invention can find many applications in a wide spectrum of fields. Each application has a specific IIED with a statement of a specific intention encoded. Without intend to limit the scope of the present invention, some examples of the specific intention statements currently undergoing test and embodiments of the present inventions are given as follows:

### Examples of Specific Intention Statements Undergoing Test

Ca⁺⁺ sparking rate in heart muscles: To activate the indwelling consciousness of X₁'s laboratory in Baltimore, Md., in order to significantly raise the EM gauge symmetry state of the interior of an electrically grounded Faraday cage (hereinafter “FC”) located in that laboratory. The special characteristic of this “conditioned” space is to be such that single, isolated, rat heart muscle cells placed inside this FC for a period of two hours develop a two-fold increase in Ca⁺⁺ sparking rate during an immediately subsequent measurement period of about two hours.

IL-6 secretion rates in a particular cell line: To activate the indwelling consciousness of X₂'s laboratory in Bethesda, Md. in order to condition the laboratory to a significantly higher EM gauge symmetry state. The special characteristic of this state is to be specifically such that samples of the macrophage cell line “raw 264.7”, cultured in an incubator with a time period of about two hour exposure to this powered IIED, will strongly interact with a lipopolysaccharide (LPS) stimulation signal. The goal of this treatment is that these cell samples will then reliably and reproducibly increase the rate of interleukin-6 (IL-6) secretion by 25% within 24 hours.

Elimination of free-radical concentration from the urine of 10 people: The recipient of one of these ten IIEDs is a subject in X₃'s Missouri experiment to significantly reduce the free-radical concentration in the body fluids such as urine of these ten living subjects. The general purpose of the intention imprint for each of these ten devices is to activate the indwelling consciousness of the subject's bedroom where an electronic a device is placed and turned on in order to condition the bedroom to a significantly higher EM gauge symmetry state. The special characteristic of this state is to reduce the free-radical concentration in the bodies of the humans exposed to that “conditioned” space. The degree of change intended is that the subject's urine will become free-radical clear, as determined by the standard free-radical test.

Broadcasting general healing via ME radiation: To activate the indwelling consciousness of X₄'s office space located in McLouth, Kans. in order to significantly raise the EM gauge symmetry state of that space. The special characteristic of this state is for the specific purpose of projecting/stimulating healing to specific individuals located at various identified remote site locations. The primary purpose of this stimulation is to reduce all unnecessary stress precursors in each individual's life so that they might manifest optimal health and daily functioning at physical, emotional and mental levels, consistent with their soul's purpose for this particular lifetime experience.

Enhancing oxygen absorption into the body from the air during sleep: To activate the indwelling consciousness of X₅'s bedroom in Payson, Ariz. in order to condition it to a significantly higher EM gauge symmetry state. X₅ is a female. The special characteristic of this state is to be specifically such that her body's intake of oxygen from the air she breathes, during both awake and sleep states, is to be increased by 30% over its present value (at that altitude of 5000 feet above sea level).

Broadcasting specific “depression”-related treatments: To activate the indwelling consciousness of X₃'s designated HOLOS laboratory space in Missouri in order to significantly raise the EM gauge symmetry state of that space so that this space becomes a conditioned space. The special characteristic of this state is for the specific purpose of broadcasting, via specific intention-modulated ME radiation, to specifically identify individuals located at various identified remote site locations that are continuously scrolled through, and displayed on the screen of, a computer located in the “conditioned” space. The primary intention-information of this broadcast is (a) to significantly reduce the periodicity, magnitude and duration of depression episodes that have heretofore occurred in the lives of these identified individuals and (b) to reduce all unnecessary stress precursors in each individual's life so that they might manifest optimal health and functioning at physical, emotional and mental levels in their daily life, consistent with their soul's primary purpose for this particular life experience.

Removing the signatures of Parkinson's from a patient: To activate the indwelling consciousness of X₆'s bedroom in Lenox, Mass. in order to condition it to a significantly higher EM gauge symmetry state. X₆ is a female and suffers headaches from time to time. The special characteristic of this state is to be specifically such as to (1) reduce, as much as possible, the thermodynamic activity of free-radicals in the cells and fluids of her body, particularly in the mid-brain area, (2) restore the substantia nigra cells of the mid-brain area to a normal healthy state, (3) significantly increase the production of dopamine in her brain, (4) significantly enhance the kinetics of L-Dopa passage through the blood/brain barrier, (5) significantly reduce both the apparent density and tendon tension in her presently crippled right hand plus the general stiffness and rigidity in her overall body and (6) significantly increase the range of all her physical movements, particularly those of her neck so that her associated headaches will disappear.

Eliminating cancers in an alternative medicine clinic: To activate the indwelling consciousness of X₇'s designated Dove Clinic Cancer Patient Treatment space in Winchester, Hampshire, England in order to significantly raise the EM gauge symmetry state of that space. The special characteristics of this state is to significantly reduce and reverse the growth of all cancerous cells in the bodies of patients regularly exposed to this “conditioned” space for several hours per visit until they are cancer-free.

It is proposed by applicants that this be therapeutically accomplished by (1) lowering the hydrogen-bonding energy of all the cancerous cell's water back into the normal range for healthy cells and increasing the thermodynamic activity of the P53 tumor suppressor genes in these cancerous cells back into the healthy range, (2) significantly increasing both the pH and oxygen thermodynamic activity of all cancerous cells so that they die and (3) significantly reducing the thermodynamic activity of the following species to normal healthy levels: (a) the vascular endothelial growth factor enzyme, (b) all protease and callaginase enzymes and (c) fas-ligand, interleukin 10 and transforming growth factor beta.

Increasing human body fitness: To activate the indwelling consciousness of X₈'s bedroom in Payson, Ariz. in order to condition it to a significantly higher EM gauge symmetry state of that space. The special characteristic of this state is to be specifically such as to (a) increase the ATP/ADP ratio in the cells of the bodies of several individuals occupying the room during both awake and asleep states by about 20% and (b) increase the bodies' immune system strength by about 25%. The ultimate purpose for these changes is to cause these bodies to be more fit, more energetic and more resilient during daily life.

Xᵢ (i=1, 2, . . . 8) in the examples of specific intention statements undergoing test set forth above represents an experimenter or practitioner who conducts the test.

Without intend to limit the scope of the invention, further exemplary procedures and preliminary experimental results of the same according to the embodiments of the present invention are given below.

### Implantation Procedures

The present invention, in one aspect, relates to a medical therapeutic procedure for utilizing a specifically tuned IIED. In one embodiment, the medical therapeutic procedure includes the steps of (a) selecting a room in a medical clinic or hospital, (b) activating the specifically tuned IIED in the room to condition the room from the U(1) EM gauge symmetry state to a higher EM gauge symmetry state that has a thermodynamic potential higher than that of the U(1) EM gauge symmetry state, (c) setting up suitable equipment in the room to continuously monitor the thermodynamic potential of the conditioned room, in terms of measuring the water proton's magnetoelectrochemical potential energy, Ψ_{H}_{⁺}, and (d) placing a living subject such as a patient with a specific condition corresponding to a disease, in the conditioned room for X hours of exposure treatment each week for Y weeks until cured. During the treatment, a medical practitioner will monitor healing progress of the living subject and determine both X and Y for the living subject. The time of exposure treatment (X and Y) is associated with the magnitude of Ψ_{H}_{⁺} in the conditioned room. If the magnitude of Ψ_{H}_{⁺} in the conditioned room is below a predetermined value, the specifically tuned IIED needs to be re-imprinted. In one embodiment, a time interval for re-imprinting the specifically tuned IIED is about 3 to 12 months. Any patients with a particular malady can be treated in the conditioned room.

In another embodiment, a medical therapeutic procedure for utilizing a specifically tuned IIED includes the step of activating the specifically tuned IIED in a patient's bedroom to condition the patient's bedroom to a higher EM gauge symmetry state so that the patient obtains nightly exposure to information encoded ME radiation corresponding to the higher EM gauge symmetry state. A medical practitioner will monitor healing progress of the patient to determine how long the specifically tuned IIED will remain in the bedroom.

In another aspect, the present invention relates to a medical therapeutic procedure for utilizing a first specifically tuned IIED and a second specifically tuned IIED. The medical therapeutic procedure, in one embodiment, includes the steps of (a) selecting a room in a medical clinic or hospital, (b) activating the first specifically tuned IIED in the room to condition the room from the U(1) EM gauge symmetry state to a higher EM gauge symmetry state which has a thermodynamic potential higher than that of the U(1) EM gauge symmetry state for healing to a group of patients whose names and addresses are continuously scrolled through a computer placed in the conditioned room, (c) activating the second specifically tuned IIED in the conditioned room that tunes the conditioned room to broadcast a specific treatment intention to the group of patients according to a list of names and addresses which is scrolled continuously through the computer, (d) setting up suitable equipment in the room to continuously monitor the thermodynamic potential of the conditioned room, in terms of measuring the water proton's magnetoelectrochemical potential energy, Ψ_{H}_{⁺}, and (e) monitoring healing progress of each of the group of patients to determine a broadcasting time for each patient.

### Preliminary Experimental Results

Ca⁺⁺ sparking rate in heart muscles: To utilize a specifically tuned IIED for medical intervention in accordance with the statement of the specific intention of increasing the Ca⁺⁺ sparking rate in heart muscles by a two-fold, as stated above, four tests have been conducted for a period of six months. Doubling of the Ca⁺⁺ sparking rate has been recorded in three of the four tests. An increase of the Ca⁺⁺ sparking rate about 150% to 175% has been observed in the fourth test.

IL-6 secretion rates in a particular cell line: To utilize a specifically tuned IIED for medical intervention in accordance with the statement of the specific intention of increasing the rate of interleukin-6 (IL-6) secretion by 25% within 24 hours, as stated above, a number of tests have been conducted and statistically significant enhancements of secretion rates have been recorded in several tests.

Elimination of free-radical concentration from the urine of 10 people: utilization of a specifically tuned IIED for medical intervention in accordance with the statement of the specific intention of eliminating the free-radical concentration from the urine of 10 people, as set forth above, has been performed for 13 people in total. The preliminary data of the undergoing experiment are listed in Table 1 below.

TABLE 1
Preliminary data of free radical concentration from urine of medical
intervened people with specifically tuned IIEDs.
After Exposure to the IIED-conditioned Bedroom

| Sub- | Background | | | | | Aver- | | Change |
|---|---|---|---|---|---|---|---|---|
| ject | Initial Value | No. 1 | No. 2 | No. 3 | No. 4 | age | Δ | (%) |
| A | 5 | 2 | 1.5 | 0.75 | 3 | 1.81 | −3.19 | −64 |
| B | 2 | 1 | 3 | 0.5 | 3 | 1.87 | −0.13 | −6.5 |
| C | 2 | 3 | 2.5 | 2.5 | 1 | 2.25 | 0.25 | 12.5 |
| D | 3 | 1 | 4.5 | 3 | 0.5 | 2.25 | −0.75 | −25 |
| E | 3 | 5 | 5 | 5 | 3 | 4.5 | 1.5 | 50 |
| F | 3 | N/A | N/A | N/A | N/A | N/A | N/A | N/A |
| G | 3 | N/A | N/A | N/A | N/A | N/A | N/A | N/A |
| H | 4 | N/A | 3 | 2 | 0.5 | 1.83 | −2.17 | −54.3 |
| I | 2 | N/A | 2 | 1 | 2 | 1.66 | −0.34 | −17 |
| J | 3 | 3 | 3 | 2 | 3 | 2.75 | −0.25 | −8.1 |
| K | 3 | N/A | N/A | N/A | 4-5 | N/A | N/A | N/A |
| L | 3.5 | N/A | N/A | N/A | 4-5 | N/A | N/A | N/A |
| M | 4 | 4 | 2 | 2 | 1 | 2.25 | −1.75 | −44 |

In Table 1, each person was assigned a letter from A to M in column Subject as his or her identification. Column Background Initial Values represents initial values of the free-radical concentration from the urine samples of the 13 people before the IIED related medical intervention. While columns No. 1, No. 2, No. 3 and No. 4 correspond to four readings of the free-radical concentration from corresponding urine samples of the 13 people during the IIED related medical intervention, respectively, and an average of the four readings of the free-radical concentration for each individual is presented in column Average. Column A is a difference between the initial value and the average of the free-radical concentration for each individual. Column Change corresponds to a change percentage of the free-radical concentration after the medical intervention.

Table 1 shows that an average change rate for 9 people whose test data are available is about 16.9%.

Broadcasting general healing via ME radiation: To evaluate medical intervention by utilizing specifically tuned IIEDs, in accordance with the statement of the specific intention of reducing all unnecessary stress precursors in a living subject's life as set forth above, a psychological state anxiety index that measures living subjects' present levels of anxiety is chosen as a testing vehicle for change in the subject population which is not really depressed. The smaller the value of the state anxiety index is, the less the anxiety of a living subject has. A 2×2 mixed analysis of variance (hereinafter “ANOVA”) of the raw data yields p=0.089, which is a marginal but optimistic result for a ground-breaking new type of research.

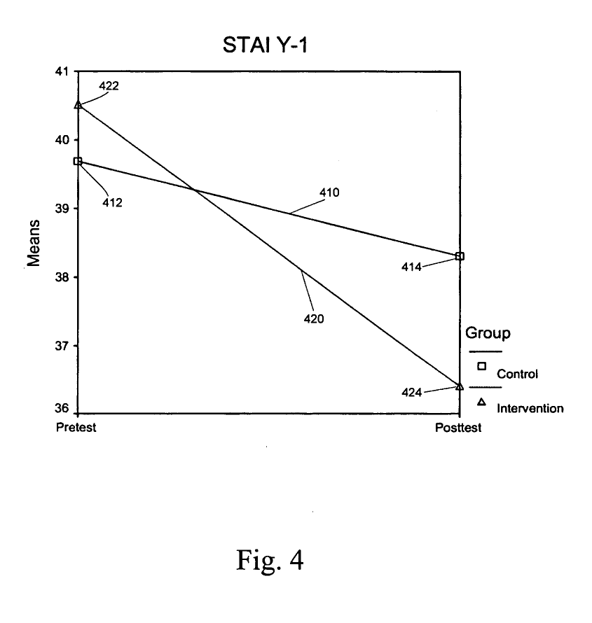

Referring now to FIG. 4, values 412 and 414 are respectively a pre-test mean value and a post-test mean value of the state anxiety index for a control group of living subjects. Line 410 connecting the pre-test mean values 412 and the post-test mean value 414 represents a trend of changes in the state anxiety index for the control group. Values 422 and 424 are respectively a pre-test mean value and a post-test mean value of the state anxiety index for a medical intervention group of living subjects. Line 420 connecting the pre-test mean values 422 and the post-test mean value 424 represents a trend of changes in the state anxiety index for the medical intervention group. It is evident that the post-test value of the state anxiety index of the medical intervention group of living subjects is less than that of the control group of living subjects, even though the medical intervention group has a pre-test mean value of the state anxiety index greater than that of the control group of living subjects. This shows that the anxiety level of the medical intervention group of living subjects has been significantly improved after the IIED-related medical intervention.

The foregoing demonstrates, among other things, that human intention can be imprinted, via a consciousness-type of process, into an intention imprinted device that can act as a surrogate for the intention to robustly influence physical reality in full accordance with the corresponding intention statement. When activated, the intention imprinted device will condition its surrounding environment (or physical space) to raise the EM gauge symmetry of the physical space to a state having a higher EM gauge symmetry than the U(1) EM gauge symmetry such that when an object (an biological object or a non-biological object) is exposed in the conditioned physical space, interaction of the object with the fields having the higher EM gauge symmetry of the conditioned physical space will produce robust physical changes in the object.

The present invention can find more applications in a wide spectrum of fields. Among them, an immediate application is to measure an EM gauge symmetry surrounding a living subject that is higher than the U(1) EM gauge symmetry, via measuring a proton magnetoelectrochemical potential energy in close proximity to the body of the living subject. Another application is to strengthen a body Qi/prana pump of a living subject so as to control a Qi-flow at high Qi-intensity. In practice, the living subject is exposed to an information encoded radiation generated from an IIED in a limited physical space for a period of time, where the limited physical space is conditioned to have at least one EM gauge symmetry higher than the U(1) gauge symmetry.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

### LIST OF REFERENCES

[1]. I. Swann, To Kiss the Earth Goodbye, (Hawthorne Books, Inc., New York, 1975).
[2]. J. Hasted, The Metal Benders, (Routledge & Kegan Paul, London, 1981).
[3]. R. Targ and H. Puthoff, Mind Reach: Scientists Look at Psychic Ability, (Delacorte Press, Eleanor Friede, New York, 1977).
[4]. D. I. Radin, The Conscious Universe: The Scientific Truth of Psychic Phenomena, (Harper Edge, San Francisco, 1997).
[5]. W. A. Tiller, Science and Human Transformation: Subtle Energies, Intentionality and Consciousness, (Pavior Publishing, Walnut Creek, Calif., 1997).
[6]. W. A. Tiller and W. E. Dibble, Jr., “New Experimental Data Revealing an Unexpected Dimension to Materials Science and Engineering”, Materials Research Innovations, 5 (2001) 21-34.
[7]. Webster's New World Dictionary, 2^{nd} College Edition (The World Publishing Company, USA, 1970 & 1972).
[8]. T. Nørretranders (J. Sydenham, Translator), The User Illusion: Cutting Consciousness Down to Size, (Penguin Books, New York, 1999).
[9]. W. A. Tiller, W. E. Dibble, Jr. and M. J. Kohane, Conscious Acts of Creation: The Emergence of a New Physics, (Pavior Publishing, Walnut Creek, Calif., 2001).
[10]. K. Moriyasu, An Elementary Primer for Gauge Theory, (World Scientific, Singapore, 1983).
[11]. T. W. Barrett, “Comments in the Harmuth Ansatz: Use of a Magnetic Current Density in the Calculation of the Propagation Velocity of Signals by Amended Maxwell Theory”, IEEE Transactions of Electromagnetic Compatibility, 30 (1988) 419.
[12]. W. E. Dibble, Jr. and W. A. Tiller, Electronic Device-Mediated pH Changes in Water, Journal of Scientific Exploration, 13, 2, (1999), pp. 155-176.
[13]. W. A. Tiller, W. E. Dibble, Jr. and M. J. Kohane, Towards Objectifying Intention via Electronic Devices, Subtle Energies and Energy Medicine, 8, 2, (1999), pp. 103-123.
[14]. M. J. Kohane and W. A. Tiller, Energy, Fitness and Information-Augmented Electromagnetic Fields in Drosophila Melanogaster, J. Sci. Expl. 14, 2 (2000) 217.

1. A method for therapeutic treatment of a living subject in a limited physical space, wherein the limited physical space is characterized by a series of symmetry states having a ground symmetry state and at least one higher symmetry state, comprising the steps of:
a. providing an intention imprinted electronic device adapted for a specific condition of the living subject;
b. activating the intention imprinted electronic device in the limited physical space for a period of time so as to transform the limited physical space from the ground symmetry state to a higher symmetry state; and
c. placing the living subject in the limited physical space for a period of time for therapeutic treatment of the specific condition of the living subject.

2. The method of claim 1, further comprising the step of keeping the intention imprinted electronic device tuned to the specific condition of the living subject for at least a portion of the period of time during which the living subject is placed in the limited space.

3. The method of claim 1, further comprising the step of monitoring response of the living subject in the limited physical space at the higher symmetry state for evaluation of the therapeutic treatment for the living subject.

4. The method of claim 1, further comprising the steps of:
a. maintaining the limited physical space at the higher symmetry state; and
b. monitoring the magnitude of a thermodynamic free energy related to the limited physical space to provide feedback to the maintaining step.

5. The method of claim 4, wherein the thermodynamic free energy of the limited physical space comprises a proton magnetoelectrochemical potential energy.

6. The method of claim 1, wherein the ground symmetry state is characterized by a U(1) electromagnetic gauge symmetry.

7. The method of claim 6, wherein the at least one higher symmetry state is characterized by a symmetry that is not a U(1) electromagnetic gauge symmetry.

8. The method of claim 7, wherein the at least one higher symmetry state is characterized by a SU(2) symmetry.

9. The method of claim 1, wherein the limited physical space comprises a space substantially confined in three dimensions.

10. The method of claim 9, wherein the confined space comprises the interior space confined by a stationary structure.

11. The method of claim 9, wherein the confined space comprises the interior space confined by a structure that is stationary or in motion.

12. The method of claim 1, further comprising the step of conditioning the limited space with an information encoded radiation generated from the intention imprinted electronic device.

13. The method of claim 12, wherein the information encoded radiation comprises an electric field signal.

14. The method of claim 12, wherein the information encoded radiation comprises a magnetic field signal.

15. An apparatus for therapeutic treatment of a living subject in a limited physical space, wherein the limited physical space is characterized by a series of symmetry states having a ground symmetry state and at least one higher symmetry state, comprising:
a. an intention imprinted electronic device adapted for a specific condition of the living subject, wherein when the intention imprinted electronic device is activated in the limited physical space for a period of time, the limited physical space is transformed from the ground symmetry state to a higher symmetry state; and
b. means for monitoring the magnitude of a thermodynamic free energy related to the limited physical space as a function of time to determine the activation time period of the intention imprinted electronic device for therapeutic treatment of a living subject in the limited physical space.

16. The apparatus of claim 15, wherein the intention imprinted electronic device comprises a single-oscillator circuit adapted for generating signals corresponding to the specific condition of the living subject.

17. The apparatus of claim 15, wherein the intention imprinted electronic device comprises a circuit having a plurality of oscillators adapted for generating signals corresponding to the specific condition of the living subject.

18. The apparatus of claim 15, wherein the monitoring means comprises a meter for measuring the magnitude of a thermodynamic free energy related to the limited physical space.

19. A method for creating an intention imprinted electronic device for therapeutic treatment of a living subject in a limited physical space, wherein the limited physical space is characterized by a series of symmetry states having a ground symmetry state and at least one higher symmetry state, comprising the steps of:
a. providing an electronic device having an electronic circuit with at least one oscillator, wherein the electronic circuit further has an input node for receiving an input voltage, a switch coupled to the input node for changing the electronic circuit between a first state and a second state, and an indication means to indicate the state of the electronic circuit, wherein the at least one oscillator and the indication means are electrically coupled to the switch, respectively; and
b. imprinting a predetermined intention into the electronic device when the electronic device is at the first state so as to create an intention imprinted electronic device, wherein the predetermined intention is designed for therapeutic treatment of a living subject in a limited physical space.

20. The method of claim 19, wherein the imprinting step comprises the steps of:
a. intentionally cleansing the electronic device;
b. broadcasting in the limited physical space a statement corresponding to the predetermined intention designed for therapeutic treatment of the living subject in the limited physical space on the cleansed electronic device;
c. interfacing the image of the cleansed electronic device and the stated predetermined intention for a first period of time to encode information corresponding to the stated predetermined intention into the electronic device to allow the at least one oscillator to generate imprint signals containing information corresponding to the stated predetermined intention;
d. intentionally sealing the electronic device for a second period of time; and
e. repeating the steps (b)-(d) till the strength of imprint signals reaches a predetermined threshold.

21. The method of claim 20, wherein the imprinting step is performed by a group of people who are capable of entering a predetermined meditative state of consciousness and focusing the predetermined meditative state for a period of time.

22. The method of claim 20, wherein the first time period is longer than the second time period.

23. The method of claim 22, wherein the first time period is in a range of about 10 to 60 minutes, and preferably in the range of about 10 to 20 minutes.

24. The method of claim 23, wherein the second time period is in a range of about 1 to 15 minutes, and preferably in the range of about 2 to 10 minutes.

25. The method of claim 19, wherein the electronic device comprises a single-oscillator.

26. The method of claim 19, wherein the electronic device comprises a plurality of oscillators electrically coupled in parallel.

27. The method of claim 19, wherein the switch is movable between an on position and an off position.

28. The method of claim 27, wherein when the switch is at the on position, the electronic circuit is in the first state.

29. The method of claim 28, wherein when the switch is at the off position, the electronic circuit is in the second state.

30. The method of claim 19, wherein the indication means comprises a light emitting diode.

31. A method for creating an intention imprinted electronic device for therapeutic treatment of at least one living subject by a group of people who are capable of entering a predetermined meditative state of consciousness, comprising the steps of:
a. providing an electronic device having an electronic circuit with at least one oscillator;
b. focusing a first predetermined meditative state of consciousness on the electronic device for a first period of time to cleanse the electronic device, the first predetermined meditative state of consciousness comprising a statement of an intention of cleansing the electronic device;
c. focusing a second predetermined meditative state of consciousness on the cleansed electronic device for a second period of time to encode information of the second predetermined meditative state of consciousness into the cleansed electronic device, the second predetermined meditative state of consciousness comprising a statement of an intention of therapeutic treatment for the at least one living subject; and
d. focusing a third predetermined meditative state of consciousness on the encoded electronic device for a third period of time to seal the encoded electronic device, the third predetermined meditative state of consciousness comprising a statement of an intention of sealing the encoded information into the encoded electronic device and minimizing leakage of the encoded information from the encoded electronic device.

32. The method of claim 31, wherein the electronic circuit includes an input node for receiving an input voltage, a switch coupled to the input node for changing the electronic circuit between a first state and a second state, and an indication means to indicate the state of the electronic circuit.

33. The method of claim 32, wherein the at least one oscillator and the indication means are electrically coupled to the switch, respectively.

34. A method for therapeutic treatment of a group of living subjects remotely from a limited physical space, wherein each of the group of living subjects is identified by a name and an address of the corresponding living subject, and the limited physical space is characterized by a series of symmetry states having a ground symmetry state and at least one higher symmetry state, comprising the steps of:
a. providing a first intention imprinted electronic device and a second intention imprinted electronic device in the limited physical space, wherein the first intention imprinted electronic device is adapted for a specific condition of the group of living subjects, and the second intention imprinted electronic device is adapted for broadcasting specific information of the limited physical space on each of the group of living subjects;
b. activating the first intention imprinted electronic device in the limited physical space for a period of time so as to transform the limited physical space from the ground symmetry state to a higher symmetry state related to the specific condition of the group of living subjects; and
c. activating the second intention imprinted electronic device in the limited physical space for a period of time so as to tune the limited physical space to broadcast the higher symmetry state on one of the group of living subjects whose name and address are presented in the limited space; and
d. presenting a name and an address of each of the group of living subjects in the limited space iteratively for therapeutic treatment of the group of living subjects.

35. The method of claim 34, further comprising the step of monitoring response of each of the group of the living subject remotely for evaluation of the therapeutic treatment for the corresponding living subject.

36. The method of claim 34, further comprising the steps of:
a. maintaining the limited physical space at the higher symmetry state; and
b. monitoring the magnitude of a thermodynamic free energy related to the limited physical space to provide feedback to the maintaining step.

37. The method of claim 36, wherein the thermodynamic free energy of the limited physical space comprises a proton magnetoelectrochemical potential energy.

38. The method of claim 34, wherein the ground symmetry state is characterized by a U(1) electromagnetic gauge symmetry.

39. The method of claim 38, wherein the at least one higher symmetry state is characterized by a symmetry that is not a U(1) electromagnetic gauge symmetry.

40. The method of claim 39, wherein the at least one higher symmetry state is characterized by a SU(2) symmetry.

41. The method of claim 34, wherein the limited physical space comprises a space substantially confined in three dimensions.

42. The method of claim 41, wherein the confined space comprises the interior space confined by a stationary structure.

43. The method of claim 41, wherein the confined space comprises the interior space confined by a structure that is stationary or in motion.

44. The method of claim 34, further comprising the step of conditioning the limited space with an information encoded radiation generated from the intention imprinted electronic device.

45. The method of claim 44, wherein the information encoded radiation comprises an electric field signal.

46. The method of claim 44, wherein the information encoded radiation comprises a magnetic field signal.

47. The method of claim 34, wherein each of the first intention imprinted electronic device and the second intention imprinted electronic device comprises an electronic circuit with at least one oscillator.

48. An apparatus for therapeutic treatment of a group of living subjects remotely from a limited physical space, wherein each of the group of living subjects is identified by a name and an address of the corresponding living subject, and the limited physical space is characterized by a series of symmetry states having a ground symmetry state and at least one higher symmetry state, comprising:
a. a display adapted for presenting a name and an address of each of the group of living subjects iteratively in the limited space;
b. a first intention imprinted electronic device adapted for a specific condition of the group of lining subjects, wherein when the first intention imprinted electronic device is activated in the limited physical space for a period of time, the limited physical space is transformed from the ground symmetry state to a higher symmetry state related to the specific condition of the group of lining subjects;
c. a second intention imprinted electronic device adapted for broadcasting the higher symmetry state on each of the group of living subjects, wherein when the second intention imprinted electronic device is activated in the limited physical space for a period of time, the limited physical space is tuned to broadcast the higher symmetry state on one of the group of the living subjects whose name and address are presented on the display in the limited space; and
d. means for monitoring the magnitude of a thermodynamic free energy related to the limited physical space as a function of time, to determine the activation time period of the first intention imprinted electronic device.

49. The apparatus of claim 48, wherein the first intention imprinted electronic device comprises a circuit with at least one oscillator.

50. The apparatus of claim 48, wherein the second intention imprinted electronic device comprises a circuit with at least one oscillator.

51. The apparatus of claim 48, wherein the monitoring means comprises a meter for measuring the magnitude of a thermodynamic free energy related to the limited physical space.

52. An apparatus for therapeutic treatment of a living subject in a limited physical space, comprising:
a. a casing defining a volume, wherein the casing is formed with a material transparent to EM signals;
b. an electronic circuit received in the casing, wherein the electronic circuit has an input node and at least on oscillator coupled to the input node; and
c. a switch formed on the casing, wherein the switch is coupled to the electronic circuit and movable between an on position and an off position such that when the switch is at the on position, a current flows through the electronic circuit and the oscillator generates and outputs EM signals to the surrounding environment through the casing, and wherein the EM signals are generated corresponding to a specific condition of the living subject.

53. The apparatus of claim 52, wherein when the switch is at the on position, a current flows through the electronic circuit and the oscillator may receive and store EM signals from the surrounding environment through the casing.

54. The apparatus of claim 52, wherein when the switch is at the off position, there is no current flows through the electronic circuit.

55. The apparatus of claim 52, wherein the electronic circuit comprises a plurality of oscillators adapted for generating signals corresponding to the specific condition of the living subject and coupled in parallel.

56. The apparatus of claim 52, wherein the current is provided by a DC source.

57. The apparatus of claim 52, wherein the current is provided by an AC source.

58. The apparatus of claim 57, further comprising an insertion opening formed on the casing adapted for receiving a conducting means to receive a current.
